(19) United States
(12) Patent Application Publication
(10) Pub. No.: US 2013/0171614 A1
(43) Pub. Date: Jul. 4, 2013

(54) EXTRACORPOREAL PERFUSION CIRCUIT SENSOR ASSEMBLY AND METHODS OF ISOLATING SENSORS THEREOF IN SEPARATE DRY AND WET ENVIRONMENTS AND STERILIZATION OF THE ASSEMBLY

(71) Applicants: Thomas L. Taccini, Denver, CO (US); Michael T. Collins, Castle Rock, CO (US); Christopher L. Jaynes, Centennial, CO (US); Peter W. Kroehl, Denver, CO (US)

(72) Inventors: Thomas L. Taccini, Denver, CO (US); Michael T. Collins, Castle Rock, CO (US); Christopher L. Jaynes, Centennial, CO (US); Peter W. Kroehl, Denver, CO (US)

(73) Assignee: XVIVO Perfusion AB, Göteborg (SE)

(21) Appl. No.: 13/817,624

(22) PCT Filed: Sep. 2, 2011
(86) PCT No.: PCT/US11/50335

§ 371 Date: Feb. 19, 2013

Related U.S. Application Data
(60) Provisional application No. 61/379,880, filed on Sep. 3, 2010.

(51) Int. Cl. A61B 5/1455; A61B 5/145; A01N 1/02; A61M 1/36

(52) U.S. Cl. CPC A61B 5/14557; A61M 1/3666; A61B 5/14556; A61B 5/14539; A01N 1/021
USPC 435/1.2; 600/312; 435/284.1

(57) ABSTRACT

Packaged cardiopulmonary bypass or organ perfusion system sensor subassemblies are provided. The packaged sensor subassemblies include a sensor subassembly and a package. The sensor subassembly includes a pre-calibrated pH sensor in a dry environment, a pre-calibrated PC0₂ sensor in a dry environment, and a pre-calibrated pC0₂ sensor in a wet environment, is sterile, and is disposed in the package. Also provided are methods of use of the packaged cardiopulmonary bypass or organ perfusion system sensor subassemblies.

### BACKGROUND OF THE INVENTION

This invention relates generally to extracorporeal perfusion circuits, and more particularly to sensor assemblies deployed in extracorporeal perfusion circuits. During use of extracorporeal circulation or cardiopulmonary bypass, continuous measurements of critical blood parameters are important for safety of the patient. It allows faster response times compared to when a blood sample is taken and brought to a blood gas analyzer. It is also more convenient for the personnel to have continuous information instead of taking intermittent blood samples. Among the important parameters to monitor are the pH and the blood gas parameters pO₂ and pCO₂. Together those parameters assist the medical professionals to evaluate the lung and circulatory function. The parameters pH, pO₂ and pCO₂ are used together to diagnose metabolic acidosis and respiratory acidosis. Continuous monitoring of pO₂ and pCO₂ can be used to minimize risk of neurological damages during cardiopulmonary bypass. PO₂ consumption monitoring can also be used to follow the anesthesia.

An available monitor that has the capability to continuously monitor pH, pO₂ and pCO₂ is the CDI™ 500 from TERUMO Cardiovascular Systems. This system however requires calibration of the sensors before use, which is a time consuming process. According to the manufacturer each calibration takes 10 minutes. The CDI™ 500 is not a true in-line monitoring system as it involves a shunt system diverting part of the flow through the analyzer. The shunting could increase hemolysis. The system is further expensive to use. A true in-line pre-calibrated monitoring system for pH, pO₂ and pCO₂ would be beneficial for extracorporeal/cardiopulmonary bypass circuits. The problem in developing such a system is that the pCO₂ sensors that are used in such a system require storage in a liquid environment to maintain factory calibration values, whereas the pO₂ and pH sensors require storage in air to maintain factory calibration values.

Blood or perfusate analysis of pH, pCO₂ and pO₂ are also used in isolated ex-vivo or in-vivo perfusion systems of tissue and organs. Such systems can for example be used to evaluate or resuscitate an explanted donor organ before transplantation or to treat an in vivo isolated organ with more aggressive treatment than can be used systemically in the patient, for example during cancer treatment. The reason to monitor pH, pCO₂ and pO₂ in isolated organ or tissue perfusion systems is to monitor metabolic activities and acidosis and to ensure that the blood gas parameters supplied to the organ are in the physiological acceptable and appropriate ranges. When a lung is perfused those parameters are also used to evaluate the functional capability of the organ. In an isolated system blood can be used as the perfusate, but the perfusate can also be a cell free solution.

Medical pO₂ and pCO₂ sensors used for measuring the partial pressure of the gas dissolved in blood or perfusate often utilize the principal of fluorescence or phosphorescence and luminescence. A light source excites electrons in the sensor and the luminescence is detected. The gas to be measured reduces the level of excited electrons in the sensor material and thereby the partial pressure of the dissolved gas correlates with the detected luminescence. The pO₂ and pH sensors are generally stable in their pre-calibrated state in an air environment. The pCO₂ sensors, however need to be stored in a bicarbonate buffered solution in order to maintain the pre-calibrated values. If CO₂ sensors are not stored in the bicarbonate buffered solution it takes hours to re-equilibrate the sensor. This makes it difficult to have a factory pre-calibrated sensor system that measures the combination of pH, pCO₂, and pO₂ in the same sensor assembly unit. In US patent application 2006/0257094 this was solved in a gas phase sensing system, primarily for the packaging industry. In U.S. Pat. No. 5,830,138 there is described an in vivo sensor using fiber optics to measure O₂, pH and CO₂. In U.S. patent application Ser. No. 12/038,583 a CO₂ sensor that does not require storage in a buffer environment to maintain its calibration values during storage is disclosed, however this sensor is not commercially available. None of these prior art references propose the use of a dual or multiple environment system for storage and sterilization of pre-calibrated sensors prior to use, within a single sensor assembly.

### SUMMARY OF THE INVENTION

In accordance with one aspect of the invention, a pre-calibrated extracorporeal perfusion circuit sensor subassembly is provided. The sensors are factory pre-calibrated and require no further calibration by the user. The assembly provides a sensor subassembly for quick incorporation into a sensor assembly placed in connection to a perfusion tube set for the reliable detection of parameters comprising pH, pO₂ and pCO₂. The sensor subassembly can be disposable. The sensor subassembly can be low cost. In a further embodiment the sensor subassembly is used in-line with a perfusion system in an extracorporeal circulation/cardiopulmonary bypass device or in an isolated tissue or organ perfusion system. Hence, the sensor subassembly is a part of the main perfusion tubing. The sensors can be isolated from one another during storage, such that one or more sensors can be stored in a liquid solution such as a bicarbonate buffer solution, while the remaining sensors can be stored in a dry environment such as air or other dry gases. Multiple separated environments can be envisioned. The sensor subassembly can be readily sterilized in a package with the sensors in their respective wet and dry environments. The separation of the sensors before use provides the possibility to have the sensors pre-calibrated with factory settings connected in series in an in-line monitoring system. The sensor environments can be separated by use of valves that are opened upon incorporation of the sensor assembly into the perfusion circuit. The sensor subassembly can be sterilized with beta or gamma radiation.

In accordance with another aspect of the invention, a main housing is provided for receipt of the sensor subassembly, such that upon fully disposing the sensor subassembly in the main housing, the sensors are automatically registered with an optic communication cable. Further, the wet environment is automatically opened to allow the free flow of fluid over the various sensors.

In accordance with another aspect of the invention, a packaged cardiopulmonary bypass or organ perfusion system sensor subassembly is provided. The packaged sensor subassembly includes a sensor subassembly and a package. The sensor subassembly includes a pre-calibrated pH sensor in a dry environment, a pre-calibrated pO₂ sensor in a dry environment, and a pre-calibrated pCO₂ sensor in a wet environment, is sterile, and is disposed in the package.

In accordance with another aspect of the invention, a method of use of a packaged cardiopulmonary bypass or organ perfusion system sensor subassembly is provided. The method includes removing the sensor subassembly from the package, disposing the sensor subassembly in a main housing, connecting the sensor subassembly in-line with a fluid circuit via connector members, actuating the sensor subassembly from a closed state to an open state to allow fluid within the fluid circuit to flow freely through the sensor subassembly, and monitoring at least one of pH, partial pressure of oxygen, and partial pressure of carbon dioxide during use in the cardiopulmonary bypass or in the isolated organ perfusion system.

### DEFINITIONS

Cardiopulmonary bypass and extracorporeal circulation are used interchangeably throughout this application.

In-line monitoring is for this application used to describe a monitoring system which monitors the parameters directly in the blood or perfusate circulation, without diverging part of the flow to circulate past the sensors.

Sensors connected in series are defined as sensors positioned in the same tube line.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 is a perspective view of an extracorporeal perfusion circuit sensor assembly in accordance with one presently preferred aspect of the invention disposed in-line in an extracorporeal perfusion circuit with the sensor assembly shown in a closed state;

FIG. 2 is a view similar to FIG. 1 with the sensor assembly shown in an open state;

FIG. 3 is a perspective view of a sensor subassembly of the assembly of FIG. 1 with the sensor subassembly shown in a closed state;

FIG. 4 is a perspective view of an individual sensor housing of the sensor subassembly with a sensor disposed therein; and

FIG. 5 is a perspective view of a housing for the sensor subassembly with the housing shown in an open position.

### DETAILED DESCRIPTION OF PRESENTLY PREFERRED EMBODIMENTS

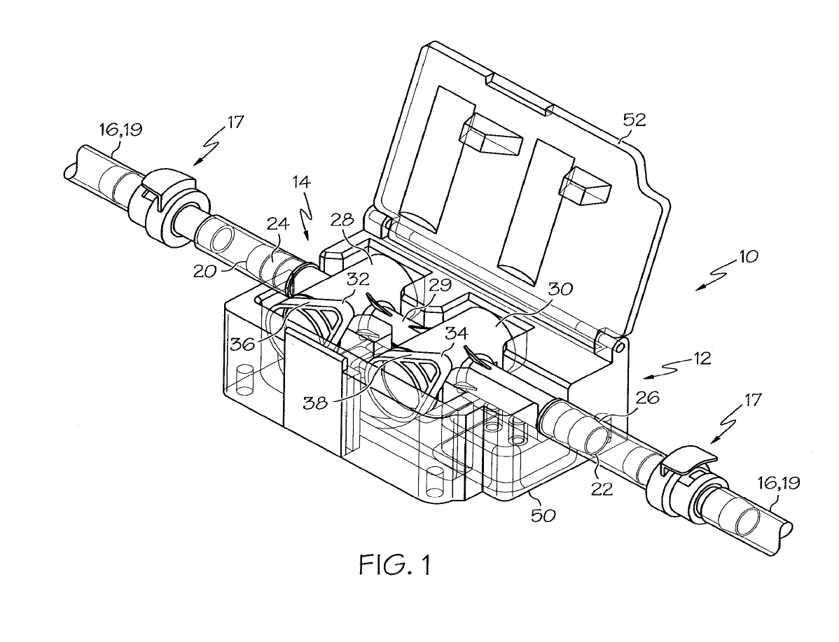

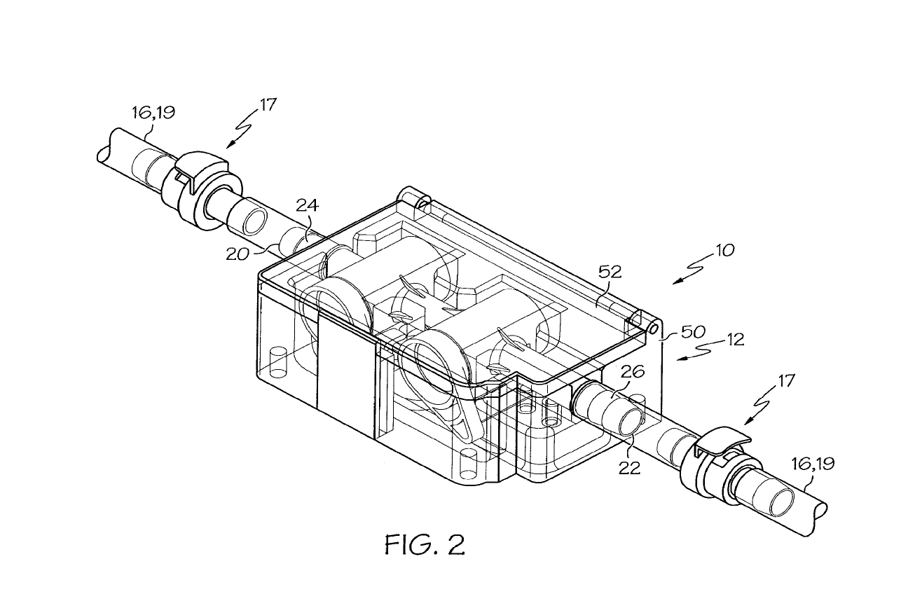

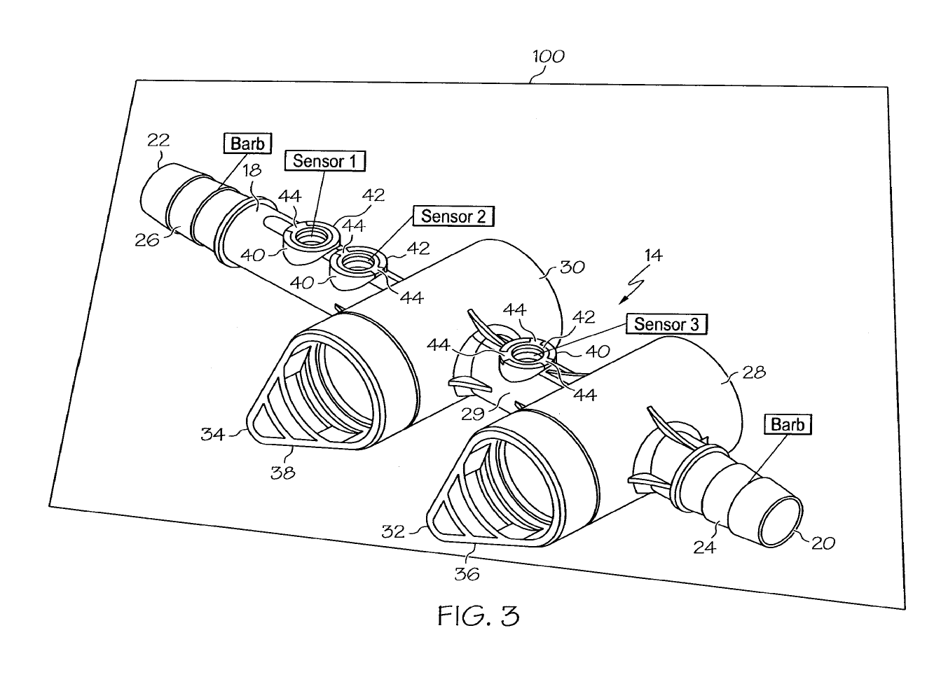

Referring in more detail to the drawings, FIGS. 1 and 2 illustrates an extracorporeal perfusion circuit sensor assembly, referred to hereafter as assembly 10, in accordance with one aspect of the invention. The assembly 10 includes a main housing 12 configured for receipt of a disposable sensor subassembly 14, wherein the sensor subassembly 14 is configured to be readily connected in-line with a disposable extracorporeal fluid circuit (a tube-set 17 of a lung transplant or cardiopulmonary bypass machine, by way of example and without limitation), referred to hereafter as fluid circuit 16, such as via quick connect/disconnect members 17. As illustrated in FIG. 3, the sensor subassembly 14 is preferably stored in a sealed package 100 (shown schematically as a solid line around the sensor subassembly 14) and sterilized after packaging, such as via gamma or beta irradiation, and maintained in its sterilized package 100 up till the desired time for use. The package 100 can be made from conventional packaging materials, including for example polyethylene. Then, when needed for use, the sterile sensor subassembly 14 is removed from its package 100, disposed in the main housing 12, connected in-line with the fluid circuit 16 via the connector members 17, and then actuated from a closed state (FIGS. 1 and 3) to an open state (FIG. 2) to allow fluid within the fluid circuit 16 to flow freely through the sensor subassembly 14. As the fluid flows through the sensor subassembly 14, the pH and/or the partial pressures of dissolved gasses (e.g. oxygen [O₂] and carbon dioxide [CO₂]) within the fluid of the perfusion circuit are continuously monitored real-time.

As best shown in FIG. 3, the sensor subassembly 14 has conduit 18 with a bidirectional fluid flow passage extending between an inlet/outlet end 20 and an outlet/inlet end 22. Both ends 20, 22 are configured for ready attachment to the fluid circuit 16, such a via respective barbed outer surfaces 24, 26 configured for fluid-tight sealed receipt within disposable the disposable tube set 19. The conduit 18 includes a pair of stopcock style valves indicated generally at 28, 30, wherein the valves 28, 30 are movable from a closed position (FIGS. 1 and 3) to an open position (FIG. 2). As such, if either one of the valves 28, 30 is in its closed position, fluid is prevented from flowing past the closed valve through the conduit 18, and if both valves 28, 30 are closed, a closed and sealed chamber 29 is established between the valves 28, 30. To facilitate opening and closing the valves 28, 30, whether done via hand or automatic actuation via the main housing 12, the valves 28, 30 have radially outwardly extending levers 32, 34, wherein each lever 32, 34 has a cam surface 36, 38 positioned in a predetermined orientation for automatic actuation of the valves 28, 30 from their closed position to their open position upon fully assembling the sensor subassembly 14 in the main housing 12 (discussed further below).

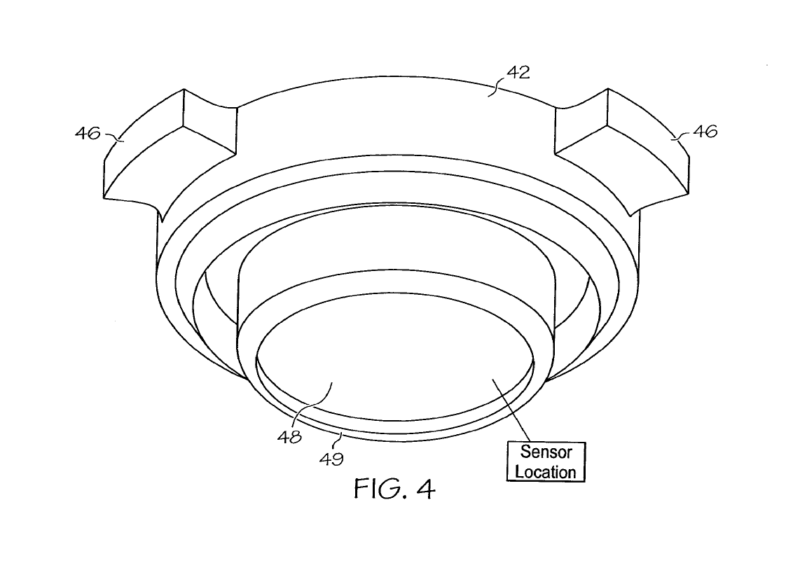

The conduit 18 further includes multiple sensor pockets, also referred to as receptacles 40. At least one of the receptacles, and shown here by way of example and without limitation as a single receptacle 40, is located between the valves 30, 32. The remaining receptacles, shown here as a pair of the receptacles 40, are located between the outlet/inlet end 22 and the adjacent valve 30. Each of the receptacles 40 is sized for receipt of a separate sensor housing 42 (FIG. 4), wherein the receptacles 40 are differentiated from one another by having a predetermined, unique pattern of key slots 44 formed about a respective periphery of the receptacles 40. As such, each receptacle 40 is unique in that it only accepts the sensor housing 42 having an outer periphery with a corresponding shape conforming with the uniquely patterned key slots 44. Accordingly, each receptacle 40 is assured of receiving therein only the intended sensor housing 42.

Each sensor housing 42 has a predetermined shape for receipt in one of the receptacles 40. To ensure the proper housing 42 is disposed in the desired receptacle 40, each housing 42 has keys 46 (FIG. 4) extending radially outwardly for receipt within a specific pattern of the key slots 44. As such, each housing 42 can only be disposed in a predetermined one of the receptacles 40, and thus, the housing 42 and a sensor 48 fixed therein can only be disposed in a predetermined receptacle 40. This assures the intended sensor 48 is positioned in the desired receptacle 40.

Each sensor 48 has a sensor spot that contains analyte-specific fluorophores. Thus, each sensor 48 is specific to detect a particular attribute of the fluid flowing through the conduit 18. For example, the pair of sensor housings 42 located between the outlet/inlet end 22 and the downstream valve 30 can house sensors 48 having sensor spots to detect hydrogen ions (pH) and O₂. Further, the sensor housing 42 located between the valves 28, 30 can house a sensor 48 having a sensor spot for detecting CO₂. Upon the sensor housings 42 and the sensors 48 therein being fixed within their associated receptacles 40, the sensors 48 are recessed in the sensor housing 42 so as not to collect bubbles, with an edge 49 facilitation retention of the sensors 48 to prevent the sensors 48 from pealing away from the their recessed position due to flow with the edge 49, wherein the sensors 48 are positioned to be within the fluid as it flows through the conduit 18. Each sensor 48 is placed in specific proximity of an optic cable (not shown) for transmission through the plastic of the detected information to an analysis mechanism (not shown).

During storage of the sensor subassembly 14, and while in its sealed and sterilized package, the stopcock valves 28, 30 are moved to their closed positions (FIGS. 1 and 3) with a liquid buffer solution sealed within the closed chamber 29. Thus, the CO₂ sensor 48 is maintained in the buffer solution while in storage and up till the time of use. As such, the CO₂ sensor 48, which are known to be extremely sensitive to damage from environmental conditions, is protected. Meanwhile, the pH and O₂ sensors 48 are stored in a dry environment outside of the sealed chamber 29. It should be recognized that the irradiation (gamma or beta) sterilization process used to sterilize the disposable sensor subassembly 14 can be performed while the sensors 48 and storage buffers are present in the subassembly 14.

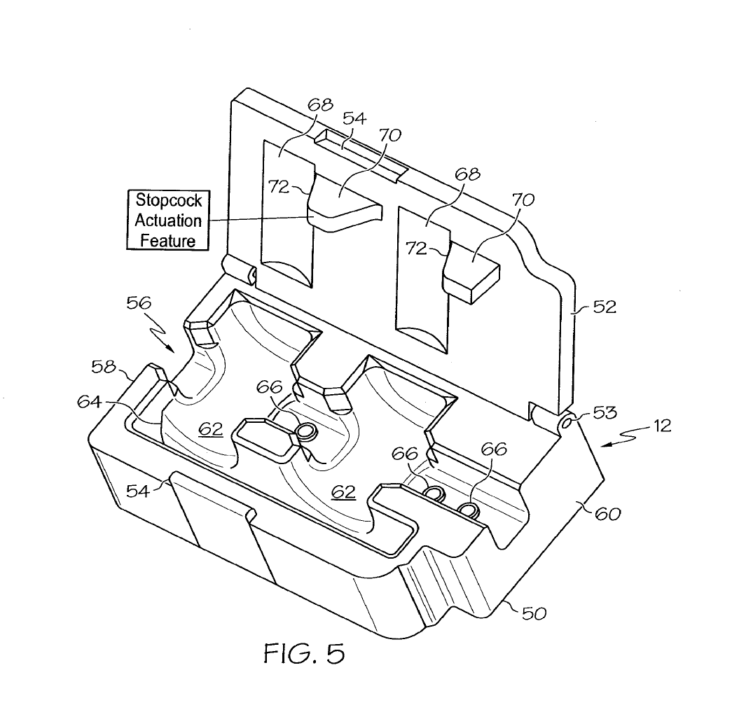

The main housing 12 of the assembly 10 is configured for receipt of the sensor subassembly 14 therein, and has unique features that automatically move the stopcock valves 28, 30 from their closed position to their open position. As best shown in FIG. 5, the main housing 12 has a base 50 and a cover 52. The cover 52 is attached to the base 50 via a hinge or hinges 53 such that the cover 52 is moveable between an open position (FIGS. 1 and 5) and a closed position (FIG. 2). When moved to the closed position, the cover 52 is locked against inadvertent opening, such as via a locking mechanism 54. Of course, the locking mechanism 54 is selectively releasable to allow the cover 52 to be opened when intended.

The base 50 has a through channel 56 extending between opposite sides 58, 60 sized for receipt of the sensor subassembly conduit 18. Further, a pair of recesses 62 extend laterally from the through channel 56 for receipt of the valves 28, 30. A further recess 64 extends along one end of the recesses 62, wherein the recess 64 is sized for receipt of the levers 32, 34, and is further sized to allow the levers 32, 34 to pivot from their closed position to their downwardly extending open position. The base further includes openings 66 positioned for alignment with the sensors 48 upon the sensor subassembly 14 being disposed in the base 50. The openings 66 are sized for receipt of the optic cables (not shown), wherein the ends of the optic cables are positioned for optical communication with the separate sensors 48.

The cover 52 is shown having a pair of recesses 68 configured for alignment with the recesses 62 in the base 50, such that the recesses 68 in the cover 52 partially receive the valves 28, 30 therein. Further, the cover 52 has a pair of actuator tabs 70. The actuator tabs 70 are positioned for engagement with the levers 32, 34 and for receipt within the recess 64 upon closing the cover 52. As such, as the cover 52 is moved from its open position (FIG. 1) toward the closed position (FIG. 2) as the actuator tabs 70 abut the lever cam surfaces 36, 38 and automatically push (actuate) the levers 32, 34 from their closed position to their open position, wherein the levers 32, 34 are pushed downwardly into the recess 64. The actuator tabs 70 each have a cam surface 72 that confronts and mates with the cam surfaces 36, 38 of the levers 32, 34 when the cover 52 is in the fully closed position, and thus, the levers 32, 34 are maintained in their open position to allow the free flow of fluid within the fluid circuit 16 through the sensor subassembly 14.

Upon completing the perfusion, the cover 52 can be opened and the sensor subassembly 14 can be disposed. The main housing 12 can then be stored and used repeatedly with a new disposable sensor subassemblies 14. Thus, the main housing 12 is reusable; secures the disposable sensor subassembly 14 during use; automatically aligns the optic cables with the appropriate sensors 48, and when the cover 52 is closed, automatically opens the stopcock valves 30 allowing fluid from the disposable fluid circuit 16 to flow over all the sensors 48 present.

### Example 1

Two Yorkshire male domestic pigs (25-35 kg) were sacrificed for isolated organ perfusion studies. The lungs were removed from the donor animals following standard lung recovery procedures (hypothermic flush with Perfadex®) and placed in cold (ice) storage during transportation. Upon arrival at the test site, the lungs were removed from the hypothermic container and placed in a sterile basin for temporary storage. The pulmonary artery (“PA”) and left atrium (“LA”) were cannulated. The perfusion tubing from the disposable lung circuit was connected to the lungs by way of straight ⅜″ hose connectors. An extracorporeal circulation device was used to provide circulation of the isolated lungs. Two sensor assemblies were connected to the lung. One was connected before the pulmonary artery and one after the out-let of the left atrium to measure pH, pCO₂ and pO₂ in both the incoming and outgoing perfusate in the lung.

In the first experiment the sensor assembly measurements from the PA and LA during the perfusion were compared to measurements from a GEM Hospital blood gas analyzer, confirming accuracy of the sensor assembly. Measurements were compared at three time points during the perfusion.

| | Time | PA pH | LA pH | PA pCO2 | LA pCO2 | PA pO2 | LA pO2 |
|---|---|---|---|---|---|---|---|
| Sensor assembly | 18:00 | 7.61 | 7.52 | 11 | 14 | 74 | 117 |
| GEM | 18:02 | 7.60 | 7.5 | 12 | 14 | 73 | 118 |
| Sensor assembly | 18:30 | 7.22 | 7.36 | 24 | 20 | 58 | 537 |
| GEM | 18:31 | 7.23 | 7.35 | 24 | 22 | 57 | 542 |
| Sensor assembly | 20:00 | 7.21 | 7.31 | 23 | 22 | 59 | 116 |
| GEM | 20:02 | 7.21 | 7.32 | 22 | 23 | 57 | 114 |

In the second experiment the sensor assembly was compared to an iSTAT POS blood gas analyzer, confirming accuracy of the sensor assembly.

| | Time | PA pH | LA pH | PA pCO2 | LA pCO2 | PA pO2 | LA pO2 |
|---|---|---|---|---|---|---|---|
| Sensor assembly | 11:30 | 7.25 | 7.37 | 19 | 19 | 57 | 194 |
| iSTAT | 11:33 | 7.22 | 7.34 | 18 | 17 | 55 | 187 |
| Sensor assembly | 13:00 | 7.06 | 7.28 | 25 | 22 | 60 | 195 |
| iSTAT | 13:04 | 7.10 | 7.23 | 23 | 23 | 56 | 190 |

Many modifications and variations of the present invention are possible in light of the above teachings. It is, therefore, to be understood that the invention may be practiced otherwise than as specifically described, and that the scope of the invention is defined by any ultimately allowed claims.

1. A packaged cardiopulmonary bypass or organ perfusion system sensor subassembly comprising:
a sensor subassembly, and
a package;
wherein the sensor subassembly comprises a pre-calibrated pH sensor in a dry environment, a pre-calibrated pO₂ sensor in a dry environment, and a pre-calibrated pCO₂ sensor in a wet environment; is sterile; and is disposed in the package.

2. A packaged sensor subassembly according to claim 1, wherein the pH sensor, the pO₂ sensor, and the pCO₂ sensor do not require further calibration before use in cardiopulmonary bypass or isolated organ perfusion.

3. A packaged sensor subassembly according to claim 1, wherein the pH sensor, the pO₂ sensor, and the pCO₂ sensor are factory pre-calibrated.

4. A packaged sensor subassembly according to claim 1, wherein the sensors are connected in series.

5. A packaged sensor subassembly according to claim 1, wherein:
the sensor subassembly further comprises a conduit with a bidirectional flow passage, an inlet/outlet end, an outlet/inlet end, two valves, a closed and sealed chamber, and a liquid buffer solution; and
the conduit extends between the inlet/outlet end and the outlet/inlet end, the two valves are disposed along the conduit, the closed and sealed chamber is disposed between the two valves, the liquid buffer solution is in the closed and sealed chamber, and the pCO₂ sensor is maintained in the liquid buffer solution.

6. A packaged sensor subassembly according to claim 1, wherein:
the sensor subassembly is configured for receipt in a main housing, such that upon removing the sensor subassembly from the package and fully disposing the sensor subassembly in the main housing, the sensors are automatically registered with an optic communication cable and the wet environment is automatically opened to allow free flow of fluid over the pH sensor, the pO₂ sensor, and the pCO₂ sensor.

7. A method of use of a packaged sensor subassembly according to claim 1 for cardiopulmonary bypass or isolated organ perfusion comprising:
removing the sensor subassembly from the package;
disposing the sensor subassembly in a main housing;
connecting the sensor subassembly in-line with a fluid circuit via connector members;
actuating the sensor subassembly from a closed state to an open state to allow fluid within the fluid circuit to flow freely through the sensor subassembly;
and monitoring at least one of pH, partial pressure of oxygen, and partial pressure of carbon dioxide during use in cardiopulmonary bypass or isolated organ perfusion.

8. A method according to claim 7, wherein the sensor subassembly is not further sterilized before use in cardiopulmonary bypass or isolated organ perfusion.

9. A method according to claim 7, wherein the pH sensor, the pO₂ sensor, and the pCO₂ sensor are not further calibrated before use in cardiopulmonary bypass or isolated organ perfusion.

10. A method according to claim 7, wherein the monitoring is continuous and in real-time.

11. A method according to claim 7, wherein the monitoring is of pH, partial pressure of oxygen, and partial pressure of carbon dioxide.
